(19) United States
(12) Patent Application Publication
(10) Pub. No.: US 2020/0258605 A1
(43) Pub. Date: Aug. 13, 2020

(54) ELECTRONIC HEALTH RECORDS MANAGEMENT USING WIRELESS COMMUNICATION

(71) Applicant: Elaine Blechman, Boulder, CO (US)

(72) Inventor: Elaine Blechman, Boulder, CO (US)

(21) Appl. No.: 16/785,004

(22) Filed: Feb. 7, 2020

Related U.S. Application Data
(60) Provisional application No. 62/802,376, filed on Feb. 7, 2019.

(51) Int. Cl. G16H 10/60; G06K 7/10; G16H 40/67

(52) U.S. Cl. CPC G16H 10/60; G16H 40/67; G06K 7/10366

(57) ABSTRACT

A patient-controlled electronic health record (PCHR) system disclosed herein provides for positioning a wireless communication (WC)-capable mobile device next to an WC tag, identifying the WC-capable mobile device via at least one of a voice query and a text query, requesting a provider to self-identify, to acknowledge patient ownership, and future control of any disclosed information via voice or text entries and to identify the provider's ECHR, in response to receiving a required response, providing the provider immediate, time-limited access to patient demographic and health payment identifiers and key elements of the patient's health history and status that the patient or patient representative has authorized for disclosure under such circumstances.

### CROSS-REFERENCE TO RELATED APPLICATIONS

This patent application is a non-provisional patent application based on U.S. provisional patent application Ser. No. 62/802,376 filed on Feb. 7, 2019 and entitled “Electronic Health Records Management Using Wireless Communication,” which is specifically incorporated by reference herein for all that it discloses or teaches.

### FIELD

Implementations disclosed herein relate, in general, to information management technology and specifically to health records management systems.

### SUMMARY

A patient-controlled electronic health record (PCHR) system disclosed herein provides for positioning a wireless communication (WC)-capable mobile device next to an WC tag, identifying the WC-capable mobile device via at least one of a voice query and a text query, requesting a provider to self-identify, to acknowledge patient ownership, and future control of any disclosed information via voice or text entries and to identify the provider's ECHR, in response to receiving a required response, providing the provider immediate, time-limited access to patient demographic and health payment identifiers and key elements of the patient's health history and status that the patient or patient representative has authorized for disclosure under such circumstances.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Other features, details, utilities, and advantages of the claimed subject matter will be apparent from the following more particular written Detailed Description of various embodiments and implementations as further illustrated in the accompanying drawings and defined in the appended claims.

### BRIEF DESCRIPTION OF THE DRAWINGS

A further understanding of the nature and advantages of the present technology may be realized by reference to the figures, which are described in the remaining portion of the specification. In the figures, like reference numerals are used throughout several figures to refer to similar components. In some instances, a reference numeral may have an associated sub-label consisting of a lower-case letter to denote one of multiple similar components. When reference is made to a reference numeral without specification of a sub-label, the reference is intended to refer to all such multiple similar components.

FIG. 1 illustrates an example deployment diagram of the physical structure of an exclusively patient-controlled electronic health record system (PCHR) that with wireless communication tokens (PCHR WC Tokens) enforces patient control of their PCHR Records.

FIG. 2 illustrates an example data access domain model shows a conceptual data structure that assures availability to users of each patient's PCHR Record in an exclusively patient-controlled electronic health record (PCHR) system is governed by that patient's explicit authorizations and also assures that contents of all patients' PCHR Records are stored, transmitted, accessed, and audited in exact original, immutable form.

FIG. 3 illustrates an example data access workflow diagram shows how, in an exclusively patient-controlled electronic health record (PCHR) system, the data owner (patient) authorizes authenticated data consumers (users) to access the PCHR Record and how the PCHR system digitally facilitates, verifies and enforces the data owner's control over the PCHR Record.

FIG. 4 illustrates an example smartwatch workflow diagram shows how, in an exclusively patient-controlled electronic health record (PCHR) system, the data owner (patient) wears a smartwatch, which contingent upon specified biometric readings, alerts and authorizes access by data consumers to data entities in the PCHR Record resulting in audit events that digitally verify each data consumer's performance and facilitate, verify and enforce the data owner's control over the PCHR Record.

FIG. 5 illustrates an example workflow diagram shows how, in an exclusively patient-controlled electronic health record (PCHR) system, the data owner (patient) stores selected data entities without personal identifiers in a public anonymized blockchain ledger and uses PCHR Consumer Apps and PCHR WC Tokens to authorize healthcare providers and other data consumers to access, update, and analyze data entities without ceding ownership of such data and without suffering damages from disclosure of personally identified data to banks, employers, insurers and others.

FIG. 6 illustrates an example screen shot, shows how, in an exclusively patient-controlled electronic health record (PCHR) system, the data owner (patient) instantly shares selected contents of the PCHR Record via a PCHR WC Token with a helpful bystander, emergency responder or healthcare provider.

FIG. 7 illustrates an example computing system that may be used to implement the PCHR system disclosed herein.

FIG. 8 illustrates an example mobile device and communicatively connected NFC tag that may be used to implement one or more parts of a PCHR system disclosed herein.

### DETAILED DESCRIPTION

Few, if any, consumers of healthcare services in the U.S., may use personal computers and mobile phones, connected to the Internet, for exclusive control over transactions within and between the totality of their patient records lodged in the exclusively enterprise-controlled electronic health record (ECHR) systems of healthcare enterprises including professional practice offices, hospitals, urgent care centers, pharmacies, laboratories, radiology centers, and health insurance companies as well as skilled-nursing, assisted-living, rehabilitation, and long-term care facilities. Online patient portals, which offer glimpses of some information in some records in some ECHR systems, suggest the illusion of consumer control. Fragmented documentation of healthcare services in the insular ECHR systems of competing healthcare enterprises, largely inaccessible to consumers but subject to frequent illegitimate breaches, burden consumers who must physically transport their health records from one provider to another and cause needless, dangerous and costly errors by healthcare providers acting on inaccurate and incomplete patient information.

For example, a 2014 Commonwealth Fund report found that test results were unavailable at appointments for 23% of Medicare patients (more than in Australia, Canada, France, Germany, Netherlands, New Zealand, Norway, Sweden, Switzerland or the UK), so doctors order costly, duplicate tests. The U.S., therefore, remains near the bottom and Singapore is at the top in Bloomberg's 2014 annual ranking of countries with the most efficient health care, reflecting health care costs as a share of GDP and per capita income, as well as life expectancy. Healthcare enterprises, competing with each other for ownership of patient care and patient information, have resisted innovations in ECHR systems that would give consumers confidence in their ability to exercise exclusive remote control over health record transactions without adverse consequences from unauthorized breaches. A fundamental innovation in electronic health records is needed to overcome the dangerous, costly gaps in knowledge about patients characteristic of conventional, insular ECHR systems.

Advances in the technology of interoperability between ECHR systems have in theory, enabled U.S. healthcare providers to exchange information about shared patients with business associates outside their organizations and with patients themselves. In practice, the costs of operating interoperable ECHR systems and the potential loss of patient revenue to competitors have proved greater than the benefits of information exchange. The exception to this rule involves integrated provider-payer organizations that derive economies of scale by using interoperable ECHR systems to exchange patient information with providers and patients who are organization members.

Such intraorganizational interoperability benefits the few patients who receive all their lifelong care from one or two large, allied healthcare enterprises and no care from providers unaffiliated with these enterprises. The fortunate few routinely encounter familiar healthcare providers with access to comprehensive, longitudinal, multi-source patient records. The unfortunate many have no usual familiar source of care, no so-called health home, which assumes all responsibility for curating, safeguarding and, when needed, sharing lifelong patient records. Intraorganizational

A growing majority of patients are in a constant state of transition between a succession of providers, unfamiliar with them and relatively ignorant of their health histories, current status, presenting complaints and previous experiences with other healthcare providers. Therefore, a fundamental innovation in electronic health records is needed, giving patients a mechanism for instant interoperability at point of care with unfamiliar providers and their diverse devices, information systems and ECHRs while safeguarding patient ownership and control over future reuse and resale of individual elements of their personally identifiable health information (PII).

A fundamental innovation in electronic health records is also needed to overcome the diffusion of responsibility resulting when many providers deliver care to the patient, but no one provider is accountable for the outcome of the patient's care, and no one provider is accountable for maintaining the total patient record, defined as the totality of patient information to date including the patient's cross-provider and cross-organization history of healthcare encounters along with the patient-generated and provider-generated data and artifacts resulting from each encounter.

Healthcare providers may destroy patient records, depending upon state laws, within ten or less years of record creation. Therefore, a fundamental innovation in electronic health records is needed to enable consumers to incorporate their clinical and financial data and artifacts from multiple information systems and ECHR sources into total patient records before sourceinformation is purged and to control and use their multi-source, longitudinal total patient records for the rest of their lives.

At point of care, patients have no uniform mechanism for rapidly informing unfamiliar providers about their history and status in order to avoid one-size-fits-all care, medical mistakes and costly repeat testing. Therefore, a fundamental innovation in electronic health records is needed at point of care to enable unfamiliar providers to instantly visualize top-level timeline summaries of total patient records, selectively focusing on lower-level details of the patient's cross-provider and cross-organization history of healthcare encounters and of patient-generated and provider-generated data and artifacts resulting from each encounter

At point of care, patients have no uniform mechanism for receiving and retaining the results of encounters with unfamiliar providers. Therefore, a fundamental innovation in electronic health records is needed at point of care to automate and enforce bidirectional information exchange, defined as a bidirectional flow of information in which during the encounter, the provider gets relevant access to the total patient record and at the end of the encounter contributes clinical and financial results of the current encounter, such as a care plan, discharge summary, diagnostic report, invoice or claim, to the total patient record.

Unauthorized access to a continuously auto-populated and updated total patient record poses a significantly greater threat to patient and provider privacy than does unauthorized access to a fragment of patient information in an ECHR or other information system silo. Patients and providers cannot productively partner in health promotion when they fear consequences of disclosure of an aggregate of personally identifiable information. Therefore, a fundamental innovation in electronic health records is needed to protect the total patient record from disclosures not knowingly authorized by the patient or the patient's personal representative or legal proxy.

Scientific advances in genomics, targeted therapies, and data analytics, make the total patient record more valuable as the basis for shared decision making by patients and their healthcare providers than fragments of the patient information in a local ECHR or other information system silo. Therefore, a fundamental innovation in electronic health records is needed to enable patients and providers, at point of care, to leverage the total patient record for real-time decision-making and for updates to the total patient record reflecting such decisions.

When a patient has a rare or complex disorder, scientific advances in genomics, targeted therapies, and data analytics, make the total patient record particularly valuable to enterprises engaged in genomic, pharmaceutical, healthcare and environmental research. Therefore, a fundamental innovation in electronic health records is needed that creates a public profile, defined as a public anonymized profile of the total patient record. The public profile would enable a marketplace in which patients, without surrendering total patient record control, could profit from licensing selective access to their current and future total patient records.

Desperate patients and family caregivers who seek advice on social media can be victimized by bullies, liars and charlatans. Therefore, a fundamental innovation in electronic health records is needed, enabling a social media forum that identity proofs forum participants and matches them with other participants based on their public profiles.

Health insurance payers and state insurance exchanges currently base their actuarial, benefits management, and fraud detection processes on a fraction of patient information. Therefore, a fundamental innovation in electronic health records is needed so that financial transactions, defined as encounter-related financial transactions between providers and payers including claims submission and reimbursement, automatically populate the total patient record. Such an innovation would enable health insurance payers, state insurance exchanges and state benefit management authorities to maintain total patient records during and after the period of plan coverage in exchange for continuing patient-authorized access to financial transactions in total patient records. Such an innovation would enable consumers to present a total patient record identification card at point of care, defined as a device that automates bidirectional information exchange between the patient record in the provider's ECHR or other information system and the total patient record in the PCHR system.

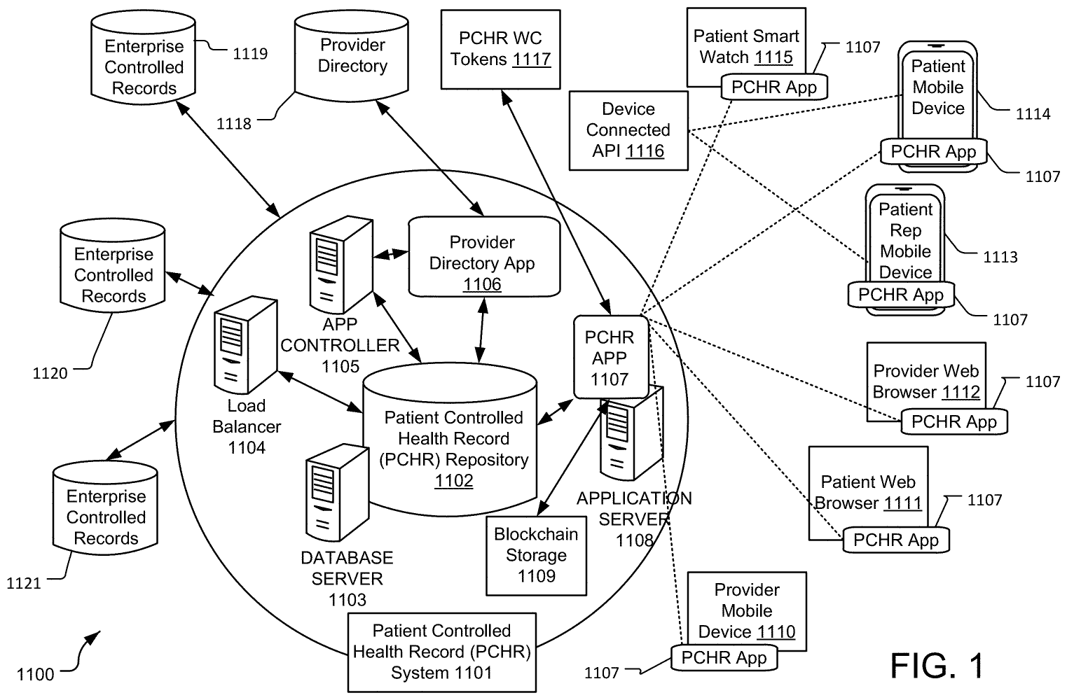

FIG. 1 is an example deployment diagram of a patient controlled electronic health record (PCHR) system 1101 for enabling patients and patient proxies via apps on web browser 1111 and mobile devices 1113, 1114, 1115 registered with the PCHR System 1101 to establish exclusively patient controlled, interoperable electronic health records (PCHR Records) and via wireless communication tokens (PCHR WC Tokens) 1117 to enforce patient control of access to their identified data stored in the data repository 1102 (PCHR Repository) and to their deidentified data stored in Blockchain Storage 1109.

The PCHR system 1101 stores patient data both with personal identifiers in PCHR Repository 1102, hosted on dedicated node Database Server 1103 and scaled as contents of PCHR Records grow, and without personal identifiers in Blockchain Storage 1109, a client of a blockchain network.

The backend application PCHR App 1107 of PCHR system 1101, which fulfills requests that patients, patient representatives and providers make from 1110, 1111, 1112, 1113, 1114 and 1115, is hosted on dedicated node Application Server 1108 and scaled as the volume of requests grow.

The App Controller 1105 manages clusters of instances 1102 and 1107 that are deployed in PCHR system 1101.

The Load Balancer 1104 distributes traffic in PCHR system 1101 so that incoming requests from 1110, 1111, 1112, 1113, 1114, 1115 and 1117 to PCHR App 1107 are distributed to Application Server 1108 nodes for request fulfillment.

PCHR App 1107 in PCHR system 1101 generates wireless communication access tokens (PCHR WC Tokens) 1117, satisfying requests made by 1111, 1113, 1114, and 1115 for sharing of data with recipients such as mobile devices and web browsers 1110, 1112 and enterprise-controlled record systems such as 1119, 1120, and 1121.

The Provider Directory App 1106 is a component of PCHR system 1101 that caches information from various external Provider Directory resources 1118 so that future requests made by 1111, 1113, 1114, and 1115 to PCHR App 1107 for retrieval of data from provider sources such as mobile devices and web browsers 1110, 1112 and Enterprise Controlled Record systems such as 1119, 1120, and 1121 can be served faster.

PCHR system 1101 interoperates by ad hoc and preconfigured means, including via device-connected Application Programming Interface API 1116, with diverse enterprise controlled record systems such as 1119, 1120, 1121 to fulfill requests that patients, patient representatives and providers make from 1110, 1111, 1112, 1113, 1114 and 1115 to PCHR App 1107 for data exchange.

In one implementation, a breast cancer survivor downloads the PCHR app 1107 to mobile device 1114, creates a PCHR Record in the PCHR Repository 1102, searches in the Provider Directory 1106 for providers from whom she has received diagnostic, treatment and aftercare services in the past and requests that they upload copies of her data from their enterprise controlled records 1119, 1120, 1121 to her PCHR Record.

In another implementation, a cancer treatment center distributes the PCHR app 1107 to patients, who from their mobile devices 1114, create PCHR Records in the PCHR Repository 1102, which the cancer treatment center populates from their enterprise-controlled records 1119, with results of their services to patients. The center refers patients for aftercare to local providers listed in the Provider Directory 1106 who authorize provider access to their PCHR Records via PCHR WC Tokens 1117.

In yet another implementation, a health insurance plan distributes PCHR Apps to its in-network providers 1111 and to its members 1114, so that members can find in-network providers in the provider directory 1118, authorize provider access to their PCHR Records at point of care via PCHR WC Tokens 1117, assure providers are fully informed about patient health status when making clinical decisions avoiding medical mistakes and upload bills for services immediately after encounters avoiding surprise bills.

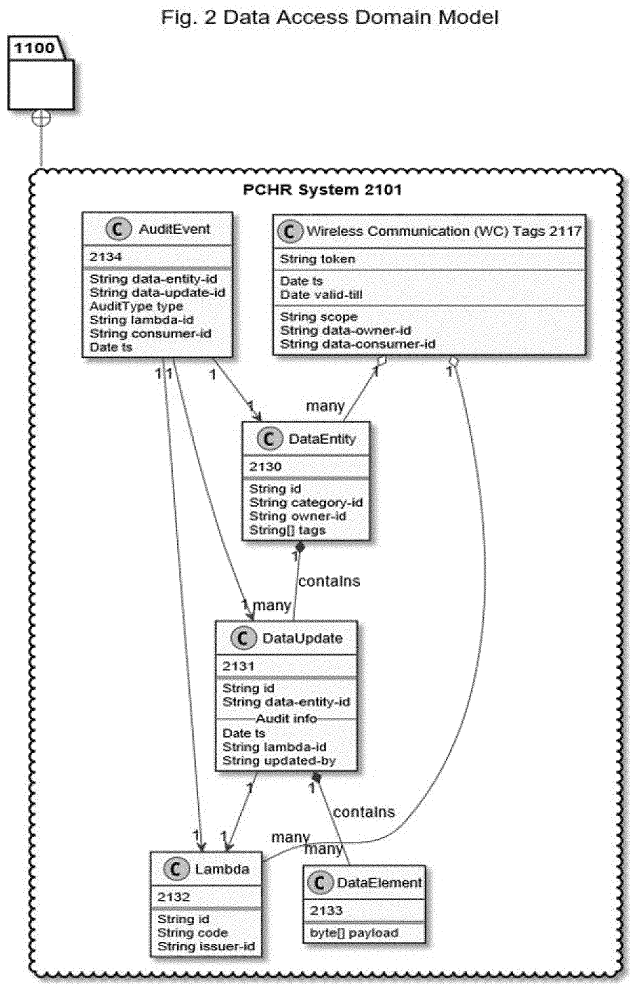

FIG. 2 illustrates an example data access domain model, shows a conceptual data structure in which wireless communication access tokens (PCHR WC Tokens) 2117 in an exclusively patient-controlled electronic health record (PCHR) system 2101 assure that availability to users of each patient's PCHR Record is governed by that patient's explicit authorizations.

Data Entity objects 2130 constitute the immutable contents of patients' PCHR Records, which in an exclusively patient-controlled electronic health record (PCHR) system 2101 are the property of patients (record owners) such that original Data Entity objects 2130 cannot be modified, original Data Entity objects 2130 can be amended through addition of Data Elements 2133 creating new Data Update objects 2131 and new Audit Events 2134, and provenance of Data Entity and Update objects 2133 and 2131 is verifiable from associated Audit Events 2134.

PCHR WC Tokens 2117 condition the time-limited availability to users of Data Entity and Data Update objects 2130 and 2131 on Lambda functions 2132 that specify and implement access authorizations of record owners and of their representatives.

Examples of Lambda 2132 include “before an encounter read and comment on care plans entered in the past month by the patient's healthcare providers,” “after an encounter upload associated test results, radiology images and diagnostic reports,” “e-prescribe medications after validation by drug reconciliation rules and transmit to patient's pharmacy,” and “run a clinical decision support algorithm, save and share results with patient and family members.”

Data Entity object 2130 may be associated with many Data Update objects 2131.

Data Update object 2131 may contain many Data Element objects 2133.

PCHR WC Tokens 2117 may be associated with a list of many Lambdas 2132

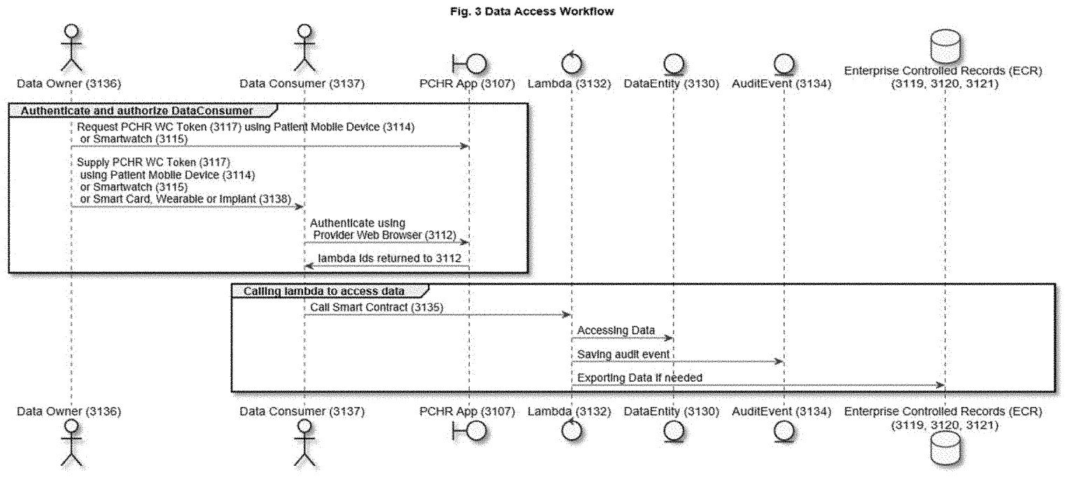

FIG. 3 illustrates an example data access workflow diagram, shows how in an exclusively patient-controlled electronic health record (PCHR) system, a data owner 3136 uses a mobile device 3114, smart watch 3115, or a smart-chipped card, wearable device or implant 3138 to instruct the PCHR App 3107 to generate a wireless communication access token (PCHR WC Token) 3117 and transmit it to the intended data consumer 3137.

The data owner 3136 uses a mobile device 3114, smart watch 3115, or smart-chipped card, wearable device or implant 3138 to transmit the PCHR WC Access Token 3117 to a data consumer 3137 who uses a mobile device 3114, web browser 3112 or dedicated scanner 3139 to receive the PCHR WC Access Token 3117.

The data consumer 3137 uses the PCHR WC Access Token 3117 to send an authentication request to the PCHR App 3107 and to request access to data entities 3130 in the PCHR Record of the data owner 3136.

If the PCHR WC Access Token 3117 is valid, the PCHR App 3107 offers the data consumer 3137 a list of Lambda 3132 (Lambda ID) options, each authorized by the data owner 3136, composed only of trusted and checked code, capable of a specific operation for accessing data entities 3130 and resulting in an audit event 3134 when executed.

Each call of a Lambda 3132 constitutes a smart contract 3135 between a data consumer 3137 and a data owner 3136 that digitally facilitates, verifies and enforces the data owner's control over the data entities 3130 in the PCHR Record and the performance of the data consumer regarding updating and sharing data entities 3130 in the PCHR Record and regarding compliance with applicable government regulations and professional practice standards without involvement of a lawyer.

In one implementation, the data owner 3136 is a patient with Parkinson's Disease whose husband 3140 uses the PCHR App 3107 on his mobile device 3113 to administers his wife's PCHR Record, establish user permissions encoded in Lambda IDs 3132 and a smart contract 3135, and transmit a PCHR WC Token 3117 to a doctor 3137 when he brings his wife for care.

In another implementation, the sister and patient representative 3140 of a patient with advanced brain cancer 3136 is notified when the patient's PCHR Record has been accessed by a surgeon 3137 under a smart contract 3135, and is connected via an encrypted communication channel with her.

In yet another implementation, a patient with multiple sclerosis 3136 transmits a PCHR WC Token 3117 under a smart contract 3135 with her primary care provider 3137, enabling him to update data entities 3130 in her PCHR Record after each encounter, to export data entities to his enterprise-controlled records 3121, and to transmit data entities to the enterprise-controlled records 3120 of a specialist 3137.

In still another implementation, a psychologist 3137 distributes the PCHR app 3107 to patients 3136, who from their mobile devices 3114, create PCHR Records in the PCHR Repository 3102, which the psychologist updates with data entities 3130 after each patient encounter and which patients authorize for transmission to behavioral-health specialists 3137 via patient-transmitted PCHR WC Tokens 3117 under smart contracts 3135 with them.

In one more implementation, a dental health insurance plan distributes PCHR Apps 3107 to its members 3136 who authorize in-network dentists 3137 to access and update x-rays and other oral health data entities in their PCHR Records via PCHR WC Tokens 3117 and smart contracts with them 3135, satisfying the patient record management needs of in-network dentists with and without enterprise-controlled record systems 3119, 3120, 3121.

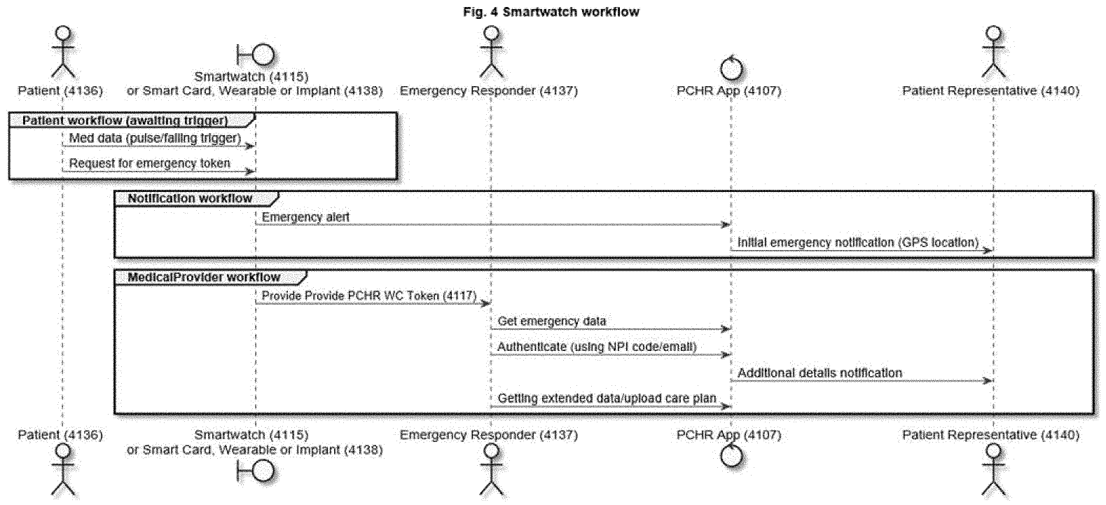

FIG. 4 illustrates an example smartwatch workflow diagram, shows how in an exclusively patient-controlled electronic health record (PCHR) system, a patient 4136 wears a smartwatch 4115 equipped with a PCHR App 4107 that initiates an emergency workflow when biometric sensors in the smartwatch and in other wearable monitors detect out-of-safe-range levels of physiological parameters that have been specified by the patient's healthcare providers, when a patient uses the PCHR App 4107 to ask for help, when the patient fails to respond to “How are you?” queries from the PCHR App 4107, when GPS coordinates indicate the patient is not in the expected geographical area or when other evidence suggests patient distress, danger or ill health.

The emergency workflow involves a pattern of activities that the PCHR App 4107 initiates, encodes as data entities 4130, enters as audit events 4134, persists and escalates until a patient 4136, patient representative 4140 or another authenticated user turns off the workflow.

One notification activity that the PCHR App 4107 initiates is display of a PCHR WC Token 4117 that when tapped or scanned by any bystander 4137 grants access to “in case of emergency” data entities in the patient's PCHR Record.

A second emergency activity that the PCHR App 4107 initiates is continuous transmission of information to the patient representative 4140 about the causes of the alert, the patient's GPS coordinates and the recipients of PCHR WC Access Tokens 4117.

A third emergency activity that the PCHR App 4107 initiates is the transmission of a request for help to previously specified or nearest available emergency response services and healthcare providers that includes PCHR WC Access Tokens 4117 and associated smart contracts 4135, GPS coordinates of patient smartwatch 4115, and a link to a secure communication channel with patient representatives 4140.

When recipients of a PCHR WC Token 4117 supply information that authenticates their credentials as licensed healthcare providers, their access to data entities in the PCHR Record increases to specialty-relevant data entities as per smart contracts 4135.

In one implementation, the data owner 4136 is a teenager with a congenital heart defect whose parents 4140 administer their son's PCHR Record, establish the user permissions encoded in Lambda IDs 4132, and equip their son with a smart watch 4115, which when biometrics exceed critical clinical cutoffs, transmits PCHR WC Tokens 4117 to nearby and emergency responders, parents, doctors, friends, teachers, and sports coaches (data consumers) 4137 enabling access suitable for each data consumer's role to data entities 4130, resulting in audit events 4134 that verify each data consumer's performance.

In another implementation involving not a watch but an implant, the data owner 4136 who serves in the military has a smart-chipped implant with biometric sensors 4138, which when biometrics exceed critical clinical cutoffs, transmits PCHR WC Tokens 4117 to individual providers authorized by the data owner 4136 and to classes of providers authorized by the military (data consumers) 4137 enabling access suitable for each data consumer's role to data entities 4130, resulting in audit events 4134 that verify each data consumer's performance.

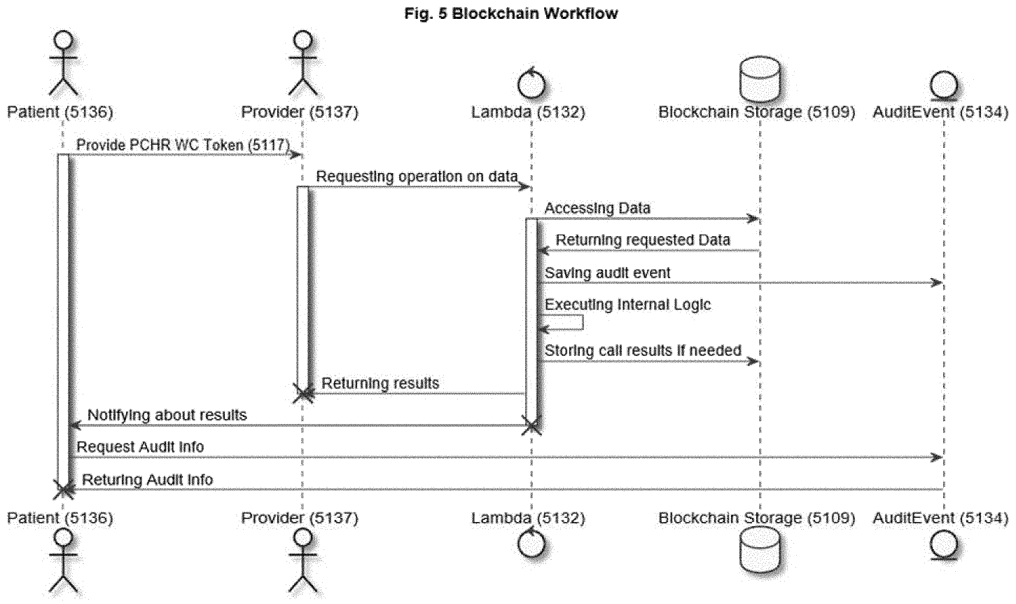

FIG. 5 illustrates an example workflow diagram, shows how in an exclusively patient-controlled electronic health record (PCHR) system, the patient 5136 may use the PCHR App 5107 on the patient mobile device 5113 to store selected data entities 5130 in the PCHR Repository with personal identifiers 5102 and without personal identifiers in blockchain storage 5109.

When the patient 5136 (or a patient representatives 5140) has reason to authorize access to identifier-free data entities 5130 in blockchain storage 5109, patient 5136 uses the PCHR App 5107 on the patient mobile device 5113 to supply PCHR WC Tag 5117 to provider 5137.

Via the PCHR App 5107 on provider web browser 5112, provider 5137 may employ PCHR WC Tag 5117 to perform operations on identifier-free data entities 5130 governed by permissions in Lambda functions 5132 established by patient 5136.

Lambda functions 5132 execute in dedicated calculation contexts with access to identifier-free data entities 5130 in blockchain storage 5109 enabling data operations permitted by patient 5136.

Provider 5137 is prohibited from performing data operations such as debugging, tracing, and accessing data entities 5130 not authorized by patient 5136 via Lambda functions 5132 encoded in PCHR WC Tag 5117.

Lambda functions 5132 access permitted data from blockchain storage 5109, report usage by generating audit events 5134, execute internal logic, such as permission checks, data filtering and throttling and searching for specific patterns in raw data, returning calculation results specified for this Lambda function 5132 to blockchain storage 5109 and to provider 5137.

PCHR App 5107 notifies patient 5136 about Lambda function 5132 call results, offers access to all generated data in blockchain storage 5109 along with the related audit event 5134 history.

In one implementation, a pediatrician 5137 diagnosed the rare genetic disorder of infant 5136 and explained to her parents 5140 that her current and future genomic, lifestyle, and treatment-response data would be so valuable to a company developing precision medicine treatments for the disorder that they might pay for the child's treatment and education. Concerned about the harmful effects of disclosure of the child's identity, with guidance by the pediatrician 5137, the child's mother 5140 used the PCHR App 5107 on her mobile device 5113 to schedule ongoing addition of selected data entities 5130 with personal identifiers to the PCHR Repository 5102 and without personal identifiers to blockchain storage 5109. Through the PCHR App, the child's mother 5140 anonymously contacts an interested pharmaceutical company 5137 and negotiates a smart contract 5135 in which the company pays to receive PCHR WC Tags 5117 that enable data operations on identifier-free data entities 5130 governed by permissions in Lambda functions 5132 and suitable for company requirements.

In another implementation, a university laboratory is conducting a clinical trial testing an investigational drug for bipolar disorder. The lab director 5137 reaches out to patients 5136 who have been diagnosed with this disorder and offers payment for study participation, anonymity, and availability to them and their future healthcare providers of data collected during the study. The lab director 5137 distributes PCHR Apps 5107 to study participants 5136 who use the PCHR App 5107 on their mobile devices 5113 to schedule ongoing addition of selected data entities 5130 with personal identifiers to the PCHR Repository 5102 and without personal identifiers to blockchain storage 5109. The lab director 5137 enters into a smart contract 5135 with study participants 5136, in which the study pays to receive PCHR WC Tags 5117 from participants 5136 that enable data operations on identifier-free data entities 5130 governed by permissions in Lambda functions 5132 as needed for study requirements.

In yet another implementation, a self-insured employer wants to reduce policy premiums by motivating employees to achieve individualized fitness goals, but without invading their privacy. The human resources (HR) manager 5137 distributes PCHR Apps 5107 to team members 5136 who use the PCHR App 5107 on mobile devices with movement and heart rate sensors 5113 to track daily fitness goals as data entities 5130 with personal identifiers in the PCHR Repository 5102 and without personal identifiers in blockchain storage 5109. The HR manager 5137 enters into a smart contract 5135 with team members 5136 who use PCHR WC Tags 5117 to transmit fitness data from blockchain storage 5109 governed by permissions in Lambda functions 5132 to earn rewards such as paid time off.

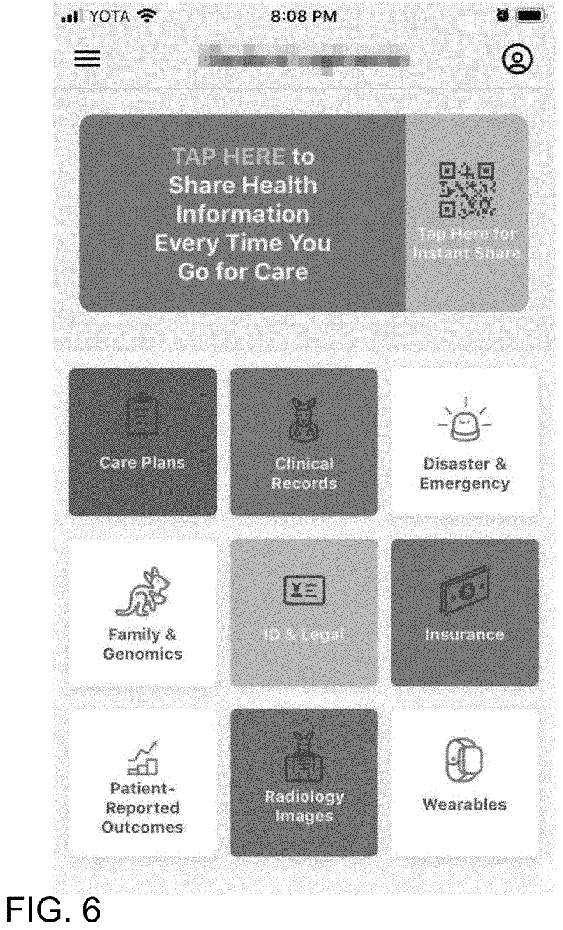

FIG. 6 illustrates an example screen shot, shows how in an exclusively patient-controlled electronic health record (PCHR) system F101, the patient F136 may use the PCHR App F107 on the patient mobile device F114 to share selected data entities F130 in the PCHR Record with a helpful bystander, emergency responder or healthcare provider F137 via a PCHR WC Token F117 configured in this example as a QR code.

The patient F136 taps on the QR code icon representing the PCHR WC Token F117 displaying a one-time, time limited QR code.

A bystander, emergency responder or healthcare provider F137 uses the camera of a mobile device to scan the QR code, authenticate identity and role, and access selected data entities F130 in the PCHR Record governed by patient permissions encoded in Lambda functions F132.

In one implementation, a patient F136 involved in an auto accident and taken to the emergency department of a nearby hospital, uses the PCHR App F107 on the patient mobile device F114 to share data entities F130 in the PCHR Record confirming health insurance coverage and usual healthcare provider, with the emergency department admitting clerk F137 who scans the PCHR WC Token QR code F117 with her mobile tablet.

In another implementation, a patient F136, in the process of getting four dental implants, has frequent visits with a general dentist, an oral surgeon, a prosthodontist and a periodontist. At the beginning of each visit, he uses the PCHR App F107 on the patient mobile device F114 to share data entities F130 in the PCHR Record such as recent dental x-rays and periodontal charts with the practice manager F137 who scans the one-time time-limited PCHR WC Token QR code F117 with her mobile tablet F113. At the end of each visit, he uses the PCHR App F107 on the patient mobile device F113 to email a one-time time limited PCHR WC Token QR code F117 to the practice manager F137 authorizing her upload of visit results as new data entities F130 to the patient's PCHR Record.

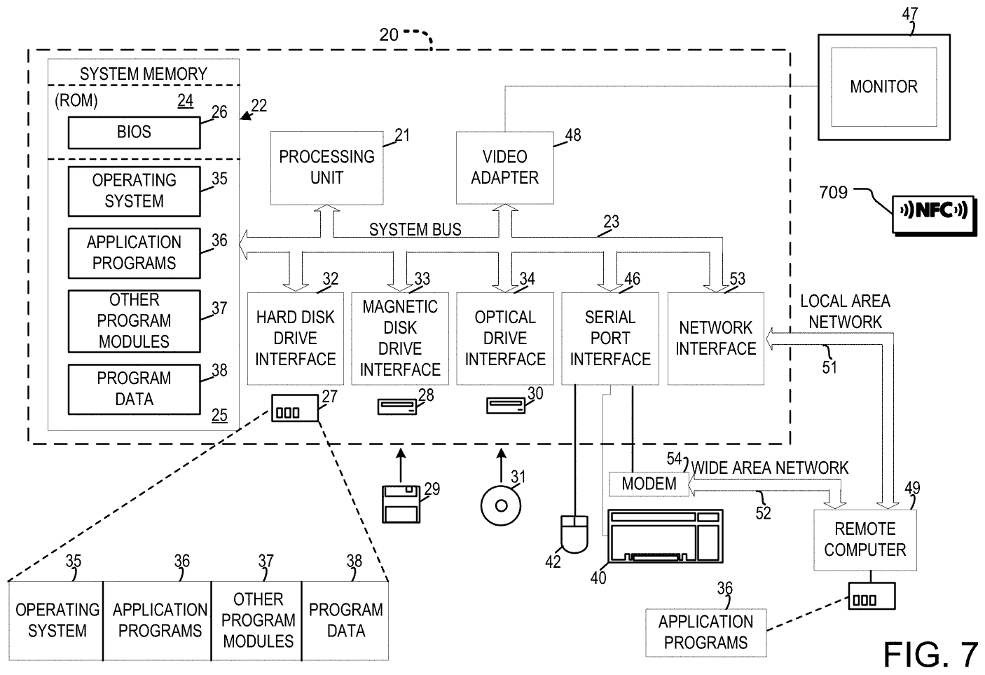

FIG. 7 illustrates an example system that may be useful in implementing the described technology. The example hardware and operating environment of FIG. 7 for implementing the described technology includes a computing device, such as general-purpose computing device in the form of a gaming console or computer 20, a mobile device, an NFC tag 2409 paired with the mobile device, a personal data assistant (PDA), a set top box, or other type of computing device. In the implementation of FIG. 7, for example, the computer 20 includes a processing unit 21, a system memory 22, and a system bus 23 that operatively couples various system components including the system memory to the processing unit 21. There may be only one or there may be more than one processing unit 21, such that the processor of computer 20 comprises a single central-processing unit (CPU), or a plurality of processing units, commonly referred to as a parallel processing environment. The computer 20 may be a conventional computer, a distributed computer, or any other type of computer; the implementations are not so limited.

The system bus 23 may be any of several types of bus structures including a memory bus or memory controller, a peripheral bus, a switched fabric, point-to-point connections, and a local bus using any of a variety of bus architectures. The system memory may also be referred to as simply the memory, and includes read only memory (ROM) 24 and random-access memory (RAM) 25. A basic input/output system (BIOS) 26, containing the basic routines that help to transfer information between elements within the computer 20, such as during start-up, is stored in ROM 24. The computer 20 further includes a hard disk drive 27 for reading from and writing to a hard disk, not shown, a magnetic disk drive 28 for reading from or writing to a removable magnetic disk 29, and an optical disk drive 30 for reading from or writing to a removable optical disk 31 such as a CD ROM, DVD, or other optical media.

The hard disk drive 27, magnetic disk drive 28, and optical disk drive 30 are connected to the system bus 23 by a hard disk drive interface 32, a magnetic disk drive interface 33, and an optical disk drive interface 34, respectively. The drives and their associated tangible computer-readable media provide nonvolatile storage of computer-readable instructions, data structures, program modules and other data for the computer 20. It should be appreciated by those skilled in the art that any type of tangible computer-readable media which can store data that is accessible by a computer, such as magnetic cassettes, flash memory cards, digital video disks, random access memories (RAMs), read only memories (ROMs), and the like, may be used in the example operating environment.

A number of program modules may be stored on the hard disk, magnetic disk 29, optical disk 31, ROM 24, or RAM 25, including an operating system 35, one or more application programs 36, other program modules 37, and program data 38. A user may enter commands and information into the personal computer 20 through input devices such as a keyboard 40 and pointing device 42. Other input devices (not shown) may include a microphone (e.g., for voice input), a camera (e.g., for a natural user interface (NUI)), a joystick, a game pad, a satellite dish, a scanner, or the like. These and other input devices are often connected to the processing unit 21 through a serial port interface 46 that is coupled to the system bus, but may be connected by other interfaces, such as a parallel port, game port, or a universal serial bus (USB). A monitor 47 or other type of display device is also connected to the system bus 23 via an interface, such as a video adapter 48. In addition to the monitor, computers typically include other peripheral output devices (not shown), such as speakers and printers.

The computer 20 may operate in a networked environment using logical connections to one or more remote computers, such as remote computer 49. These logical connections are achieved by a communication device coupled to or a part of the computer 20; the implementations are not limited to a particular type of communications device. The remote computer 49 may be another computer, a server, a router, a network PC, a client, a peer device or other common network node, and typically includes many or all of the elements described above relative to the computer 20, although only a memory storage device 50 has been illustrated in FIG. 24. The logical connections depicted in FIG. 24 include a local-area network (LAN) 51 and a wide-area network (WAN) 52. Such networking environments are commonplace in office networks, enterprise-wide computer networks, intranets and the Internet, which are all types of networks.

When used in a LAN-networking environment, the computer 20 is connected to the local network 51 through a network interface or adapter 53, which is one type of communications device. When used in a WAN-networking environment, the computer 20 typically includes a modem 54, a network adapter, a type of communications device, or any other type of communications device for establishing communications over the wide area network 52. The modem 54, which may be internal or external, is connected to the system bus 23 via the serial port interface 46. In a networked environment, program engines depicted relative to the personal computer 20, or portions thereof, may be stored in the remote memory storage device. It is appreciated that the network connections shown are example and other means of and communications devices for establishing a communications link between the computers may be used.

In an example implementation, software or firmware instructions and data for providing a search management system, various applications, search context pipelines, search services, service, a local file index, a local or remote application content index, a provider API, a contextual application launcher, and other instructions and data may be stored in memory 22 and/or storage devices 29 or 31 and processed by the processing unit 21.

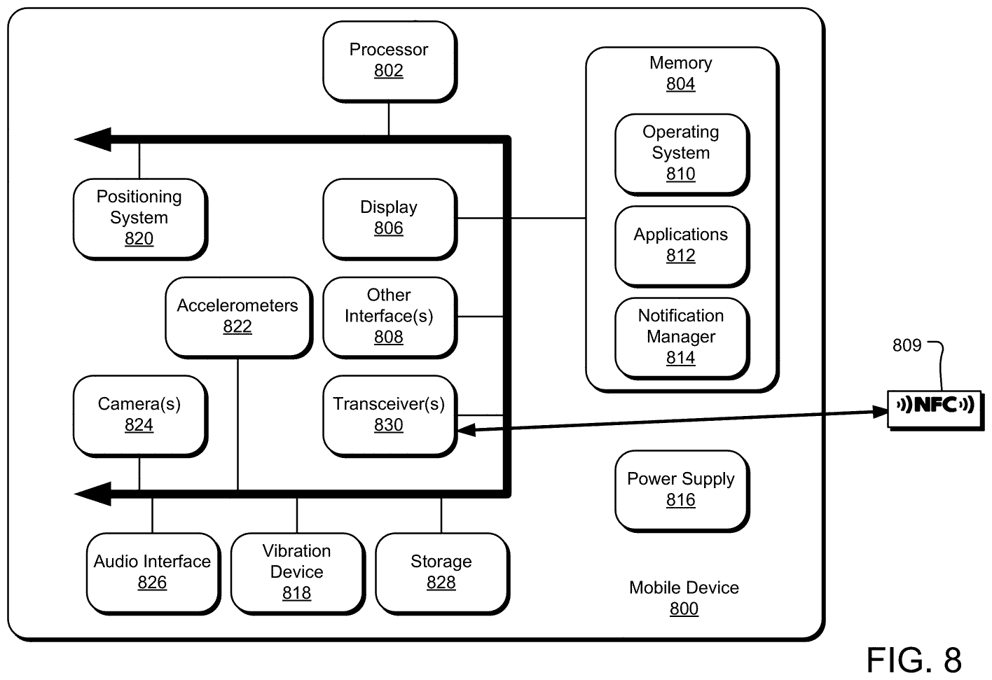

FIG. 8 illustrates another example system (labeled as a mobile device 800) that may be useful in implementing the described PCHR system technology. The mobile device 800 includes a processor 802, a memory 804, a display 806 (e.g., a touchscreen display), and other interfaces 808 (e.g., a keyboard) and a paired NFC tag 809. The memory 804 generally includes both volatile memory (e.g., RAM) and non-volatile memory (e.g., flash memory). An operating system 810, such as the Microsoft Windows® Phone 8 operating system, the Apple OS®, Android operating system, etc., resides in the memory 804 and is executed by the processor 802, although it should be understood that other operating systems may be employed.

One or more application programs 812 are loaded in the memory 804 and executed on the operating system 810 by the processor 802. Examples of applications 812 include without limitation email programs, scheduling programs, personal information managers, Internet browsing programs, multimedia player applications, etc. An implementation of the mobile device 800 may include a mobile PCHR application for the PCHR system disclosed herein, wherein the PCHR application communicates the PCHR system implemented on a cloud server, and an NFC tag 809 paired with the device. For example, such PCHR application may be configured to generate a GUI on the mobile device 800 and a paired NFC tag 809 that provides interactive capabilities to a patient using various input modalities (touch, shake, etc.).

A notification manager 814 is also loaded in the memory 804 and is executed by the processor 802 to present notifications to the user. The mobile device 800 includes a power supply 816, which is powered by one or more batteries or other power sources and which provides power to other components of the mobile device 800. The power supply 816 may also be connected to an external power source that overrides or recharges the built-in batteries or other power sources.

The mobile device 800 includes one or more communication transceivers 830 to provide network connectivity (e.g., mobile phone network, Wi-Fi®, BlueTooth®, etc.). The mobile device 800 also includes various other components, such as a positioning system 830 (e.g., a global positioning satellite transceiver), one or more accelerometers 832, one or more cameras 834, an audio interface 826 (e.g., a microphone, an audio amplifier and speaker and/or audio jack), and additional storage 828. Other configurations may also be employed.

In an example implementation, an electronic health records system, and other modules and services may be embodied by instructions stored in memory 804 and/or storage devices 828 and processed by the processing unit 802. The master pages, the layouts, and other data may be stored in memory 804 and/or storage devices 828 as persistent datastores.

A method disclosed herein includes establishing an exclusively patient controlled cloud health record (PCHR Record) in a patient controlled electronic health record system (PCHR System) by means of apps on mobile devices, smart watches and web browsers registered with the PCHR System (PCHR Apps) to patients (data owners), patient proxies and representatives (data administrators), and patient-authorized providers that with wireless communication access tokens (PCHR WC Tokens) enforce secure patient control of immutable contents of PCHR Records during and after data capture, storage with and without personal identifiers, and data consumer access (FIG. 1); storing contents of PCHR Records as exact original immutable data entities together with audit events documenting their provenance (FIG. 2); embedding data entities, data entity updates and associated audit events in unique programs executable only with data owner permissions contingent upon PCHR WC Tokens (FIG. 2); assuring that capture and update of data entities in PCHR Records from external sources is governed by data owner permissions via PCHR WC Tokens (FIG. 3); assuring that access of data consumers to data entities in PCHR Records and transport to external record systems is governed by data owner permissions via PCHR WC Tokens (FIG. 3); creating smart contracts that, without third party involvement, digitally facilitate, verify and enforce the control of data owners over contents of PCHR Records and the performance of data consumers in updating, transmitting and reselling contents of PCHR Records and in compliance with applicable government regulations and professional practice standards (FIG. 3); embedding PCHR WC Tokens and biometric sensors into an App on a PCHR Smart Watch worn by or implanted in a data owner so that, under emergency conditions established by data owners or administrators in PCHR Consumer Apps, the PCHR Smart Watch App transmits alerts to data consumers, grants access to contents of PCHR Records and collects information resulting from encounters between data owners and data consumers (FIG. 4); storing selected data entities without personal identifiers in a public anonymized blockchain ledger and with identifiers in the PCHR data repository so that data linkage requires the consent of the data owner conveyed via PCHR WC Tokens (FIG. 5); and enabling data owners via PCHR Consumer Apps and PCHR WC Tokens to authorize data consumers to access, update, and analyze data entities in the blockchain ledger without ceding ownership of such data and without suffering damages from disclosure of personally identified data to banks, employers, insurers and others (FIG. 5).

An alternative implementation provides a method including establishing an exclusively patient controlled electronic health record (PCHR Record) in the data repository (PCHR Repository) of a patient controlled, interoperable electronic health record system (PCHR System) via apps on mobile devices and computers registered with the PCHR System to patients and to patient proxies (PCHR Consumer Apps) that with wireless communication tags (PCHR WC Tags) enforce patient control of access to their PCHR Records; employing identity authentication methods to confirm the identity of patients, patient proxies, and users authorized by patients and patient proxies for PCHR Record access (Authorized Users); controlling the import of information into the PCHR Record from multiple external human and machine sources via PCHR Consumer Apps and PCHR WC Tags; storing contents of a PCHR Record in the PCHR Repository as exact original tamper-proof PCHR Data Elements together with metadata about the sources and circumstances of import to the PCHR Record; supplying options for authorized users to add comments, updates and corrections to each PCHR Data Element; controlling the export of information from the PCHR Record to multiple external human and machine recipients via PCHR Consumer Apps and PCHR WC Tags; attaching to each PCHR Data Element in the Repository, a hashtag (PCHR Data Element Hashtag) that is altered each time a comment, update or correction is added to the Data Element and each time the Data Element is accessed or exported; attaching to each patient, patient proxy and Authorized User in the Repository, a hashtag (PCHR User Hashtag) and unique identifier; storing PCHR Data Element and PCHR User Hashtags without personal identifiers in a public anonymized blockchain PCHR Ledger with links to related Data Elements and Users in the PCHR Repository; maintaining tamper-proof PCHR Audit Logs that automatically record each occurrence in a PCHR Record and are accessible to patients and patient proxies from PCHR Apps; including private keys in PCHR Apps so patients, patient proxies and Authorized Users can access, share, contribute and lease PCHR Data Elements in the anonymized blockchain PCHR Ledger without ceding ownership of valuable genomic and other PCHR Data Elements and without suffering damages from disclosure of identified PCHR Data Elements to banks, employers, insurers and others; employing PCHR Data Element Hashtags so patients, patient proxies, patient advocates and other Authorized Users may authenticate the chain of custody for PCHR Data Elements without disclosure of identified PCHR Data Element; and maintaining tamper-proof PCHR Audit Logs that automatically record each occurrence in a PCHR Record and are accessible to Authorized Users from PCHR Apps.

Yet alternatively, a method disclosed herein includes Generating unique wireless communication tags (PCHR WC Tags) via PCHR Consumer Apps on mobile devices and computers registered with the PCHR System of patients and patient proxies; displaying PCHR WC Tags on mobile devices of patients or patient proxies so that scanning of Tags, consistent with preferences established by patients or patient proxies in PCHR Consumer Apps, transmits alerts to third parties and grants access to contents of PCHR Records; embedding PCHR WC Tags in devices worn and carried by patients or implanting Tags under the skin so that, consistent with preferences established by patients or patient proxies in PCHR Consumer Apps, scanning of devices transmits alerts to third parties and grants access to contents of PCHR Records; integrating PCHR WC Tags and biometric sensors into a PCHR Consumer App for smart watches so that sensor detection of physiological data indicating high patient risk, consistent with preferences established by patients or patient proxies in PCHR Consumer Apps, transmits alerts to third parties and actionable information to mobile devices of nearby individuals; operating PCHR WC Tags so that consistent with preferences established by patients or patient proxies in PCHR Consumer Apps, when persons other than Authorized Users (Unauthorized Users) utilize mobile devices or web-connected computers to scan Tags or otherwise attempt access to a patient's PCHR Record, notifications are automatically sent to patient, patient proxy and Authorized Users who can permit or deny access to the Record; operating PCHR WC Tags so that consistent with preferences established by patients or patient proxies in PCHR Consumer Apps, when Authorized Users utilize apps on their mobile devices or web-connected computers (PCHR Provider Apps) to rapidly scan Tags and access a patient's PCHR Record, notifications are automatically sent to patient, patient proxy and Authorized Users who can permit or deny access to the Record; attaching to each transaction between and among a PCHR WC Tag, a PCHR Record, Authorized and Unauthorized Users, a unique hashtag (PCHR Transaction Hashtag); and storing PCHR Transaction Hashtags without personal identifiers in a public anonymized blockchain PCHR Ledger with links to related WC Tags, Data Elements and Users in the PCHR Repository.

Alternatively, the method includes Configuring a web portal for patient-authorized access to a PCHR Record via mobile device or web-connected computer by Authorized and Unauthorized Users who scan a PCHR WC Tag or are notified by the PCHR App on a patient's smart watch; enabling Authorized Users whose identities have been authenticated and who have written agreements with patients and patient proxies about the terms and condition of PCHR Record access to present credentials, rapidly authenticate their identities, and access patient-authorized content of the PCHR Record; requiring Unauthorized Users, under circumstances that do not pose serious threats to patient safety to authenticate their identity before accessing patient-authorized content of the PCHR Record; permitting Authorized and Unauthorized Users, who verbally affirm serious threats to patient safety, to bypass identity authentication, “break-the-glass” and access the patient's PCHR Record consistent with prevailing best practices for emergency responders; creating smart contracts that digitally facilitate, verify and enforce the adherence of users of the PCHR Record to specific data use and payment protocols; attaching to each smart contract a unique hashtag (PCHR Smart Contract Hashtag); storing PCHR Smart Contract Hashtags without personal identifiers in a public anonymized blockchain PCHR Ledger with links to related WC Tags, Transactions, Data Elements and Users in the PCHR Repository; employing PCHR Smart Contract Hashtags so patients, patient proxies, patient advocates and Authorized and Unauthorized Users may monitor fulfillment of the smart contract without disclosure of identified PCHR Data Elements; configuring smart contract protocols that enable Authorized and Unauthorized Users to exchange information with PCHR Records under circumstances that do or do not pose serious threats to patient safety in accordance with applicable rules, regulations and practice standards; and notifying patient proxies that the tag has been scanned, providing GPS coordinates for location of the scanning device and enabling User and patient proxies to communicate with each other via phone, text or chat; prompting the provider, by means of voice or text queries, to affirm in voice or text responses, that the provider, has personally reviewed contents of the patient's PCHR record (indicating which contents the provider reviewed such as medications, allergies, immunizations, problems, care plans); has noted potential safety risks reflected in the patient's PCHR record (indicating which risks the provider noted such as drug allergies); agrees to deliver services to the patient (personally and via notification of all other care team members) that are consistent with established guidelines for the patient's care reflected in contents of the patient's PCHR record (indicating which guidelines the provider noted such as the current schedule for medication administration); agrees to change any established guidelines for the patient's care reflected in contents of the patient's PCHR record only after documenting in the patient's PCHR record, the nature of the guideline changes and the written or oral consent of the patient or the patient's representative to the guideline change.

Alternatively, the method includes Prompting the provider, by means of voice or text queries, to affirm in voice or text responses, that the provider agrees to notify all personnel involved in delivery of services to the patient about contents of the patient's PCHR record (indicating which contents the provider reviewed such as medications, allergies, immunizations, problems, care plans); wherein the potential safety risks being reflected in the patient's PCHR record (indicating which risks the provider noted such as drug allergies); delivery of services to the patient (personally and via notification of all other care team members) that are consistent with established guidelines for the patient's care reflected in contents of the patient's PCHR record (indicating which guidelines the provider noted such as the current schedule for medication administration); and changes in any established guidelines for the patient's care reflected in contents of the patient's PCHR record that the provider has documented in the patient's PCHR record, the nature of the guideline changes and the written or oral consent of the patient or the patient's representative to the guideline change.

Some embodiments may comprise an article of manufacture. An article of manufacture may comprise a tangible storage medium to store and execute logic. Examples of a storage medium may include one or more types of computer-readable storage media capable of storing electronic data, including volatile memory or non-volatile memory, removable or non-removable memory, erasable or non-erasable memory, writeable or re-writeable memory, and so forth. Examples of the logic may include various software elements, such as software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. In one embodiment, for example, an article of manufacture may store executable computer program instructions that, when executed by a computer, cause the computer to perform methods and/or operations in accordance with the described embodiments. The executable computer program instructions may include any suitable type of code, such as source code, compiled code, interpreted code, executable code, static code, dynamic code, and the like. The executable computer program instructions may be implemented according to a predefined computer language, manner or syntax, for instructing a computer to perform a certain function. The instructions may be implemented using any suitable high-level, low-level, object-oriented, visual, compiled and/or interpreted programming language.

The implementations described herein are implemented as logical steps in one or more computer systems. The logical operations may be implemented (1) as a sequence of processor-implemented steps executing in one or more computer systems and (2) as interconnected machine or circuit modules within one or more computer systems. The implementation is a matter of choice, dependent on the performance requirements of the computer system being utilized. Accordingly, the logical operations making up the implementations described herein are referred to variously as operations, steps, objects, or modules. Furthermore, it should be understood that logical operations may be performed in any order, unless explicitly claimed otherwise or a specific order is inherently necessitated by the claim language.

The above specification, examples, and data provide a complete description of the structure and use of exemplary implementations. Since many implementations can be made without departing from the spirit and scope of the claimed invention, the claims hereinafter appended define the invention. Furthermore, structural features of the different examples may be combined in yet another implementation without departing from the recited claims.

Embodiments of the present technology are disclosed herein in the context of a patient-controlled electronic health record (PCHR) system. In the above description, for the purposes of explanation, numerous specific details are set forth in order to provide a thorough understanding of the present invention. It will be apparent, however, to one skilled in the art that the present invention may be practiced without some of these specific details. For example, while various features are ascribed to particular embodiments, it should be appreciated that the features described with respect to one embodiment may be incorporated with other embodiments as well. By the same token, however, no single feature or features of any described embodiment should be considered essential to the invention, as other embodiments of the invention may omit such features.

In the interest of clarity, not all of the routine functions of the implementations described herein are shown and described. It will, of course, be appreciated that in the development of any such actual implementation, numerous implementation-specific decisions must be made in order to achieve the developer's specific goals, such as compliance with application—and business-related constraints, and that those specific goals will vary from one implementation to another and from one developer to another.

According to one embodiment of the present invention, the components, process steps, and/or data structures disclosed herein may be implemented using various types of operating systems (OS), computing platforms, firmware, computer programs, computer languages, and/or general-purpose machines. The method can be run as a programmed process running on processing circuitry. The processing circuitry can take the form of numerous combinations of processors and operating systems, connections and networks, data stores, or a stand-alone device. The process can be implemented as instructions executed by such hardware, hardware alone, or any combination thereof. The software may be stored on a program storage device readable by a machine.

According to one embodiment of the present invention, the components, processes and/or data structures may be implemented using machine language, assembler, C or C++, Java and/or other high level language programs running on a data processing computer such as a personal computer, workstation computer, mainframe computer, or high performance server running an OS such as Solaris® available from Sun Microsystems, Inc. of Santa Clara, Calif., Windows Vista™, Windows NT®, Windows XP PRO, and Windows® 2000, available from Microsoft Corporation of Redmond, Wash., Apple OS X-based systems, available from Apple Inc. of Cupertino, Calif., or various versions of the Unix operating system such as Linux available from a number of vendors. The method may also be implemented on a multiple-processor system, or in a computing environment including various peripherals such as input devices, output devices, displays, pointing devices, memories, storage devices, media interfaces for transferring data to and from the processor(s), and the like. In addition, such a computer system or computing environment may be networked locally, or over the Internet or other networks. Different implementations may be used and may include other types of operating systems, computing platforms, computer programs, firmware, computer languages and/or general-purpose machines; and. In addition, those of ordinary skill in the art will recognize that devices of a less general-purpose nature, such as hardwired devices, field programmable gate arrays (FPGAs), application specific integrated circuits (ASICs), or the like, may also be used without departing from the scope and spirit of the inventive concepts disclosed herein.

In the context of the present invention, the term “processor” describes a physical computer (either stand-alone or distributed) or a virtual machine (either stand-alone or distributed) that processes or transforms data. The processor may be implemented in hardware, software, firmware, or a combination thereof.

In the context of the present technology, the term “data store,” also referred to by the term “repository,” describes a hardware and/or software means or apparatus, either local or distributed, for storing digital or analog information or data. The term “data store” describes, by way of example, any such devices as random access memory (RAM), read-only memory (ROM), dynamic random access memory (DRAM), static dynamic random access memory (SDRAM), Flash memory, hard drives, disk drives, floppy drives, tape drives, CD drives, DVD drives, magnetic tape devices (audio, visual, analog, digital, or a combination thereof), optical storage devices, electrically erasable programmable read-only memory (EEPROM), solid state memory devices and Universal Serial Bus (USB) storage devices, and the like. The term “data store” also describes, by way of example, databases, file systems, record systems, object oriented databases, relational databases, SQL databases, audit trails and logs, program memory, cache and buffers, and the like.

The above specification, examples and data provide a complete description of the structure and use of exemplary embodiments of the invention. Although various embodiments of the invention have been described above with a certain degree of particularity, or with reference to one or more individual embodiments, those skilled in the art could make numerous alterations to the disclosed embodiments without departing from the spirit or scope of this invention. In particular, it should be understood that the described technology may be employed independent of a personal computer. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A method, comprising;
establishing an exclusively patient controlled health record (PCHR Record) in a patient controlled electronic health record system (PCHR System) by means of apps on one or more of a mobile device, a smart watch, and a web browser registered with the PCHR System (PCHR Apps) to patients (data owners), patient proxies and representatives (data administrators), and patient-authorized providers that with wireless communication access tokens (PCHR WC Tokens) enforce secure patient control of immutable contents of PCHR Records during and after data capture, storage with and without personal identifiers, and data consumer access;
storing contents of PCHR Records as exact original immutable data entities together with audit events documenting their provenance;
embedding data entities, data entity updates and associated audit events in unique programs executable only with data owner permissions contingent upon PCHR WC Tokens;
assuring that capture and update of data entities in PCHR Records from external sources is governed by data owner permissions via PCHR WC Tokens;
assuring that access of data consumers to data entities in PCHR Records and transport to external record systems is governed by data owner permissions via PCHR WC Tokens;
creating smart contracts that, without third party involvement, digitally facilitate, verify and enforce the control of data owners over contents of PCHR Records and the performance of data consumers in updating, transmitting and reselling contents of PCHR Records and in compliance with applicable government regulations and professional practice standards;
embedding PCHR WC Tokens and biometric sensors into an App on a PCHR Smart Watch worn by or implanted in a data owner so that, under emergency conditions established by data owners or administrators in PCHR Consumer Apps, the PCHR Smart Watch App transmits alerts to data consumers, grants access to contents of PCHR Records and collects information resulting from encounters between data owners and data consumers;
storing selected data entities without personal identifiers in a public anonymized blockchain ledger and with identifiers in the PCHR data repository so that data linkage requires the consent of the data owner conveyed via PCHR WC Tokens; and
enabling data owners via PCHR Consumer Apps and PCHR WC Tokens to authorize data consumers to access, update, and analyze data entities in the blockchain ledger without ceding ownership of such data and without suffering damages from disclosure of personally identified data to banks, employers, insurers and others.

2. The method of claim 1, further comprising generating unique wireless communication tags (PCHR WC Tags) via a PCHR consumer apps on mobile devices and computers registered with the PCHR system of a patient and patient proxies.

3. The method of claim 2, further comprising displaying the PCHR WC tags on mobile devices of patients or patient proxies such that scanning of the PCHR WC tags, consistent with preferences established by the patient or the patient proxies in the PCHR consumer apps, initiates transmitting alerts to third parties and grants access to contents of the PCHR records to the third parties.

4. The method of claim 2, further comprising embedding the PCHR WC tags in devices worn and carried by patients or implanting the PCHR WC tags under patient skin so that, consistent with preferences established by the patient or the patient proxies in the PCHR consumer apps, scanning of devices initiates transmitting of alerts to third parties and grants access to contents of the PCHR records to the third parties.

5. The method of claim 2, further comprising integrating the PCHR WC tags and biometric sensors into the PCHR consumer app for smart watches including a sensor so that the sensor's detection of physiological data indicating high patient risk, consistent with preferences established by the patient or the patient proxies in PCHR consumer app, initiates transmitting of alerts to third parties and grants access to contents of the PCHR records to the third parties.

6. The method of claim 2, further comprising operating the PCHR WC tags so that, consistent with preferences established by the patient or the patient proxies in PCHR consumer apps, in response to a person other than authorized users utilizing mobile devices or web-connected computers to scan the PCHR WC tags, notifications are automatically sent to the patient, the patient proxies, and the authorized users.

7. The method of claim 6, further comprising attaching a PCHR transaction hashtag to a transaction between or among a PCHR WC tag, a PCHR record, and authorized or unauthorized users.

8. The method of claim 7, further comprising storing the PCHR transaction hashtag without any personal identifiers in a public anonymized blockchain PCHR Ledger.

9. The method of claim 8, further comprising storing the PCHR transaction hashtag without any personal identifiers in a public anonymized blockchain PCHR Ledger with links to related to the PCHR WC tags, data elements, and users in a PCHR repository.

10. A method, comprising:
positioning a wireless communication (WC)-capable mobile device next to an WC tag;
identifying the WC-capable mobile device via at least one of a voice query and a text query;
requesting a provider to self-identify, to acknowledge patient ownership, and future control of any disclosed information via voice or text entries and to identify the provider's ECHR; and
in response to receiving a required response, providing the provider immediate, time-limited access to patient demographic and health payment identifiers and key elements of the patient's health history and status that the patient or patient representative has authorized for disclosure under such circumstances.

11. The method of claim 10, further comprising:
capturing the wherein the WC tag captures the provider's mobile device identification information, relays it to the registered device of the patient representative, and thereby to the patient's PCHR record, where it is stored permanently in an audit trail. The PCHR record captures information about the provider's ECHR preferences in anticipation of future data exchanges.

12. The method of claim 10, wherein the WC tag is a near field communication (NFC) tag.
